(19) United States
(12) Patent Application Publication
(10) Pub. No.: US 2017/0303876 A1
(43) Pub. Date: Oct. 26, 2017

(54) WEARABLE X-RAY IMAGING APPARATUS AND METHOD FOR USING THE SAME

(71) Applicants: VATECH Co., Ltd., Gyeonggi-do (KR); VATECH EWOO Holdings Co., Ltd., Gyeonggi-do (KR)

(72) Inventors: Hyung Keun LIM, Gyeonggi-do (KR); Jin Pyo CHUN, Gyeonggi-do (KR)

(73) Assignees: VATECH Co., Ltd., Gyeonggi-do (KR); VATECH EWOO Holdings Co., Ltd., Gyeonggi-do (KR)

(21) Appl. No.: 15/511,449

(22) PCT Filed: Sep. 15, 2015
(86) PCT No.: PCT/KR2015/009674

§ 371 Date: Mar. 15, 2017

(30) Foreign Application Priority Data
Sep. 15, 2014 (KR) 10-2014-0121794

(51) Int. Cl. A61B 6/14; A61B 6/00; A61B 6/10

(52) U.S. Cl. CPC A61B 6/145; A61B 6/4405; A61B 6/14; A61B 6/107

(57) ABSTRACT

The present invention relates to a wearable X-ray radiographing apparatus and a method for using the same. The present invention includes: an X-ray generator including an X-ray source, a power supplier supplying driving power to the X-ray source, and a cone directing X-rays emitted from the X-ray source; a switch controller controlled by a radiographer to operate the X-ray generator; and a wearable unit on which the X-ray generator being mounted and being worn on the body of the radiographer.

### TECHNICAL FIELD

The present invention relates to an X-ray radiographing apparatus. More particularly, the present invention relates to a wearable X-ray radiographing apparatus in which a movable X-ray radiographing apparatus is mounted to the body of a radiographer for radiographing.

### BACKGROUND ART

Among intraoral radiographing apparatuses, movable X-ray radiographing apparatuses are widely used in a variety of fields such as medical and industrial fields due to their small size, light weight, low cost, and ease of use.

FIG. 8 is a view showing a conventional movable X-ray radiographing apparatus in use, and shows X-ray radiographing of teeth and tissues around the teeth within the mouth cavity by using a movable X-ray radiographing apparatus that is used in the dental field.

As shown in FIG. 8, in a method for radiographing an oral cavity using a conventional movable X-ray radiographing apparatus 100, a radiographer holds the movable radiographing apparatus 100 with both hands, disposes a sensor within the oral cavity of an examinee, and radiographs the oral. In other words, the sensor is mounted in the dental cavity by using a sensor arrangement device (indicator), and radiographing is performed by placing an X-ray cone of the X-ray radiographing apparatus on an irradiating area that is indicated outside the dental cavity.

However, when there is difficulty in arranging the sensor by coupling the indicator, the radiographer sometimes performs radiographing by holding the sensor with one hand while gripping the X-ray radiographing apparatus with the other hand. When the radiographing apparatus is manipulated with one hand as described above, there is a problem that the handling of the radiographing apparatus becomes unstable due to the basic weight and volume of the apparatus even though the movable radiographing apparatus is small and light weight. In addition, when the radiographer grips the radiographing apparatus with one hand, there is a problem that it is difficult to immediately respond to emergency situations.

In addition, there is a problem that waiting time of the examinee increases according to a moving path of the radiographer to prepare the apparatus after mounting the sensor in the oral cavity.

### DISCLOSURE

### Technical Problem

The present invention has been made keeping in mind the above problems occurring in the prior art, and an object of the present invention is to provide a wearable X-ray radiographing apparatus whereby stability of a radiographer's posture may be ensured, radiographing waiting time of an examinee may be reduced, and a damage to the radiographing apparatus caused by being dropped while in use may be prevented by enabling a movable X-ray radiographing apparatus to be worn on the body of a radiographer.

In addition, another object of the present invention is to provide a wearable X-ray radiographing apparatus capable of preventing the radiographer from being exposed to radiation.

In addition, a further object of the present invention is to provide a method for using the wearable X-ray radiographing apparatus.

### Technical Solution

In order to accomplish the above object, a wearable X-ray radiographing apparatus according the present invention includes: an X-ray generator including an X-ray source, a power supplier supplying driving power to the X-ray source, and a cone directing X-rays emitted from the X-ray source; a switch controller controlled by a radiographer to operate the X-ray generator; and a wearable unit on which the X-ray generator being mounted and the wearable unit being worn on a body of the radiographer

According to a wearable X-ray radiographing apparatus according of a first embodiment of the present invention, in the configuration of the X-ray radiographing apparatus described above, the switch controller may be physically separated from the X-ray generator and connected thereto in a wired or wireless manner. In other words, the X-ray radiographing apparatus according to the first embodiment may be configured such that the X-ray generator is worn on the body of the radiographer, and the radiographer holds the switch controller by hand and operates the switch controller by pressing it.

The wearable unit may include: a holder holding the X-ray generator; and a body configured with a shielding material to be worn on the body of the radiographer

In addition, in the X-ray generator of the X-ray radiographing apparatus, the X-ray source may include the power supplier supplying a driving voltage to the X-ray source or a connection terminal to receive power from an external source, and the X-ray source may further include a user controller to control the X-ray generator.

Meanwhile, according to a wearable X-ray radiographing apparatus of a second embodiment of the present invention, in the configuration of the X-ray radiographing apparatus described above, the X-ray generator may include an irradiator and a controller that are physically separated from each other and are connected to each other in a wired or wireless manner, wherein the irradiator includes the X-ray source and the cone, the controller includes the power supplier, and the wearable unit includes: at least one of a first wearable unit on which the irradiator is mounted and which is worn on a first body part of the radiographer and a second wearable unit on which the controller is mounted and which is worn on a second body part of the radiographer. Herein, the irradiator includes the X-ray source and the cone, the controller includes the power supplier. In addition, the switch controller may be physically separated from the irradiator and connected thereto in a wired or wireless manner.

In other words, the X-ray radiographing apparatus of the second embodiment may be configured such that the irradiator and the controller of the X-ray generator are respectively worn on the first body part and the second body part by the radiographer, and the radiographer holds the switch controller by hand and operates the switch controller by pressing it.

In addition, according to a wearable X-ray radiographing apparatus according a third embodiment of the present invention, as the second embodiment, as the second embodiment, the X-ray generator is configured to includes the irradiator and the controller, but the switch controller operating the X-ray generator is mounted on the irradiator. In other words, the X-ray radiographing apparatus according to the third embodiment may be configured such that the controller is worn on the body of the radiographer, the radiographer grips the irradiator on which the switch controller is mounted and presses the switch controller to operate the irradiator.

In addition, the X-ray radiographing apparatus according to the first to the third embodiments may further include a shielding membrane provided around the cone.

Meanwhile, according to another aspect of the present invention, a method for using the wearable X-ray radiographing apparatus according to the first to the third embodiments includes: wearing the wearable unit on the body of the radiographer whereby the cone is placed on a hand part of the radiographer; and arranging the cone on a radiographing part of a target, and irradiating X-rays on the radiographing part of the target through the cone by operating the X-ray generator by using the switch controller.

### Advantageous Effects

According to the wearable X-ray apparatus and the method for using the same according to the above present invention, there is provided an effect of ensuring the stability of a radiographer's posture and reducing radiographing waiting time of an examinee by enabling the movable X-ray radiographing apparatus to be worn on the body of the radiographer. In addition, it provides an advantage of preventing the radiographing apparatus from being dropped while in use and allows the radiographer to immediately respond to unexpected emergency situations.

In addition, according to the X-ray radiographing apparatus of the present invention, there is provided an advantage that radiation exposure to the radiographer may be prevented more easily and reliably than in a conventional X-ray radiographing apparatus by configuring the wearable unit of the X-ray generator that is worn on the body of the radiographer with a shielding material.

### DESCRIPTION OF DRAWINGS

FIG. 1 is a view showing a configuration of a wearable X-ray radiographing apparatus according to a first embodiment

FIG. 2 is a block diagram showing a schematic configuration of an X-ray generator 100 of the X-ray radiographing apparatus of FIG. 1.

FIG. 3 is a view showing a configuration of a wearable X-ray radiographing apparatus according to a second embodiment of the present invention.

FIG. 4 is a block diagram showing a schematic configuration of an X-ray generator of the wearable X-ray radiographing apparatus according to the second embodiment of the present invention.

FIG. 5 is a view showing a schematic configuration of a wearable X-ray radiographing apparatus according to a third embodiment of the present invention.

FIG. 6 is a view schematically showing an embodiment in which a shielding membrane is provided at an end part of a cone of the wearable X-ray radiographing apparatus according to embodiment of the present invention.

FIG. 7 is a view schematically showing another embodiment in which the shielding membrane is provided at the end part of the cone of the wearable X-ray radiographing apparatus according to embodiment of the present invention.

FIG. 8 is a view showing a conventional movable X-ray radiographing apparatus in use.

### DESCRIPTION OF THE REFERENCE NUMERALS

100 X-ray generator 110 Cone

120 Housing of X-ray generator 120-1 Irradiator

120-2 Controller 130 Switch controller

140 Shielding membrane 121 X-ray source

200 Wearable unit 210 Body

220 Holder

### MODE FOR INVENTION

Hereinbelow, exemplary embodiments of the present invention will be described in detail with reference to the accompanying drawings. Although embodiments of a wearable X-ray radiographing apparatus according to the present invention are disclosed for intraoral X-ray radiographing, the scope of the present invention is not limited thereto. Further, the scope of the present invention may be widely used in related fields such as industrial, medical, and the like.

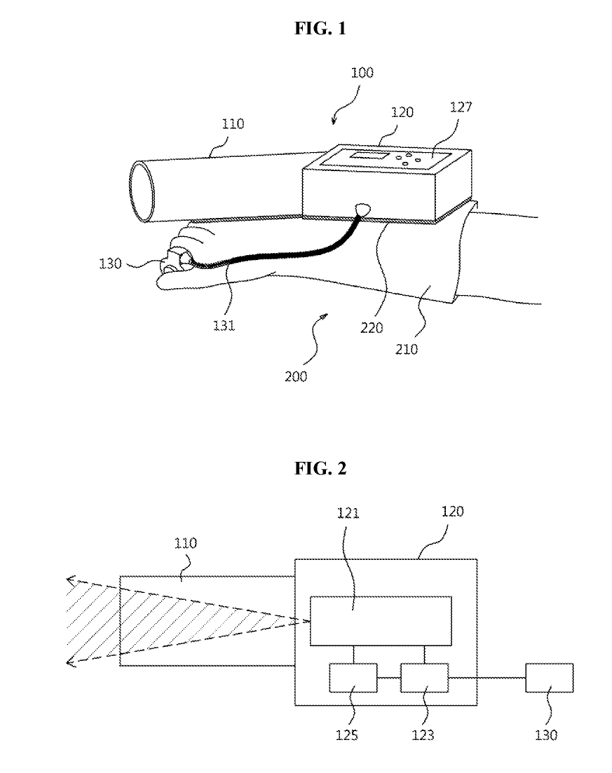

First, as shown in FIG. 1, a wearable X-ray radiographing apparatus according to a first embodiment of the present invention may be configured to include an X-ray generator 100, a switch controller 130 controlling an operation of the X-ray generator 100, and a wearable unit 200 on which the X-ray generator 100 is mounted and which is worn on a body of a radiographer.

The X-ray generator 100 includes a housing 120 in which an X-ray source 121 and a cone 110, which directs X-rays irradiated from the X-ray source 121 to a specific direction, are provided as schematically shown in an exterior of FIG. 1.

In detail, as shown in FIG. 2, the X-ray source 121 that generates X-rays, a power supplier 125 supplying a driving voltage to the X-ray source 121, and a controller 123 that generates a control signal to control each part of the X-ray generator 100 may be provided inside the housing 120.

The X-ray source 121 that generates X-rays may be applied in a limited area of a movable X-ray generating device. It is preferable that the X-ray source 121 to be as small as possible and generates X-rays with high efficiency at a low power. Thus, as the X-ray source 121, a thermo-electronic X-ray tube that generates X-rays by colliding electrons emitted by high-temperature heat of a tungsten filament against a target may be used. However, it is preferable to use a field emission type X-ray tube using a nano-structured material that generates X-rays by colliding electrons emitted from the nano-structured material such as carbon nanotubes, etc.

In the X-ray generator 100, the power supplier 125 may be provided in a limited area of the movable X-ray generating device and supply the driving voltage thereto, and any power source known in the art may be used without limitation. The power supplier 125 includes a configuration of raising a voltage of a power supplying device and a voltage supplied therefrom to a high voltage that is finally used in the X-ray source. In addition, the power supplying device, for example, may be a conversion device that converts a voltage of a battery or a voltage supplied therefrom, or may be replaced with a connection terminal to receive power from an external source.

In the first embodiment of the present invention, since the X-ray generator 100 is worn on the body of the radiographer, it is preferable that the size and weight of the X-ray generator 100 is reduced for convenience of radiographing by configuring the power supplier 125 to include a connection terminal in order to receive power from an external source.

Meanwhile, in the X-ray radiographing apparatus according to the first embodiment, the switch controller 130 operating the X-ray generator 100 is formed to be physically separated from the X-ray generator 100. Herein, the switch controller 130 that is physically separated from the X-ray generator 100 may be connected to the X-ray generator 100 in a wired or wireless manner. Although not particularly limited, considering interference with a wireless signal generated by the X-rays, it is preferable to wirily connect the switch controller 130 to the X-ray generator 110 using a cable 131. The controller 123 generates a signal controlling an operation of the X-ray source 121 according to a signal of the switch controller 130 that is independently formed from the X-ray generator 100 as described above.

In addition, a user controller 127 is provided in the X-ray generator 100 to set a control mode of the X-ray source 121. The controller 123 may generate a control signal to operate the X-ray source 121 in the control mode selected through the user controller 127. For example, the control mode that may be selected through the user controller 127 may include a radiographing patient such as adult, child, etc., a radiographing target such as anterior tooth, eyetooth, molar tooth, etc., a radiographing form such as film, digital radiography (DR), etc., and options related to an output and an operation of the X-ray source 121 that may be set by a user for X-ray radiographing according to a radiographing object such as applying voltage such as tube voltage, applying current such as tube current, X-ray exposure time, etc.

Detailed descriptions of a detailed operational description of the X-ray generator 100 except for the configuration described above and an intraoral radiographing method for a target will be omitted since it is the same as a general movable intraoral x-ray radiographing apparatus usually used in the technical field.

Referring again to FIG. 1, the X-ray radiographing apparatus of the present invention includes the wearable unit 200 on which the X-ray generator 100 is mounted and which is worn on a body of the radiographer. In detail, the wearable unit 200 includes a holder 220 that holds the X-ray generator 100 and a body 210 that is worn on the body of the radiographer.

The holder 220 is configured to couple the X-ray generator 100 and the body 210 of the wearable unit 200 that is worn on the body of the radiographer. One side of the holder 220 is configured to be mounted on the body 210 and the other side of the holder 220 is configured to mount the X-ray generator 100.

In detail, the above configuration on which the X-ray generator 100 is mounted, as shown in FIG. 1 as an example, a storage space in which a protrusion having a predetermined height is formed according to a contacting boundary of the X-ray generator 110 is formed, thus the X-ray generator 100 is configured to be fitted in and dismounted from the storage space. However, there is no particular limitation on the configuration of the holder (storage space), the body 210 of the wearable unit 200 and the X-ray generator 100 may be coupled with each other by using a belt and a buckle, or various locking means such as a Velcro tape that is capable of locking two different structures may be applied without limitation. In addition, if necessary, the holder 220 may permanently fix the X-ray generator 100 and the body 210 so that the X-ray generator 100 and the body 210 are not separated from each other.

In addition, the body 210 is worn on the body of the radiographer, and considering that the apparatus is for intraoral X-ray radiographing, it is preferable to form and configure the body 210 to be worn by radiographer at a location from his/her forearm (lower part of the arm) to his/her hand. In detail, the body 210 may be configured with a gauntlet or in a band configuration to be worn on the hand and the forearm of the radiographer as shown in the figure.

Meanwhile, the X-ray generator 100 may be mounted on a part of the body 210 that is worn on by the radiographer at any location from his/her forearm to a part of a back of his/her hand, or may be mounted on a part that is worn on a lateral side of the radiographer's forearm to a part of the hand near his/her the thumb, thereby in the above two cases the cone 110 of the X-ray generator 110 is mounted on a hand part of the wearable unit 200.

A part or the entire part of the body 210, and preferably at least a part that is adjacent to the X-ray generator 100, may be formed of a material capable of shielding X-rays, or the body itself may be made of X-ray shielding fiber. By constituting the body 210 with the shielding material as described above, it is possible to more easily and reliably prevent the radiographer from being exposed to radiation during the operation of the X-ray generator 100.

A usage method for the X-ray radiographing apparatus according to the first embodiment will be described.

First, the wearable unit 200 of the X-ray radiographing apparatus is worn by a radiographer at a location from his/her forearm (lower part of the arm) to his/her hand. When the wearable unit 200 is configured with a gauntlet, the wearable unit 200 is worn by the radiographer inserting his/her hand into the wearable unit 200 and by pulling up the wearable unit 200 to his/her forearm just like putting on a glove. By being worn as described above, the cone 110 of the X-ray generator 100 is placed on the hand part of the radiographer.

In detail, depending on where the X-ray generator 100 is mounted on the wearable unit 200, the X-ray generator 100 is worn by a radiographer on a part from an outside of his/her forearm to a back his/her hand, or on a part from a lateral side of his/her forearm to his/her thumb.

When the X-ray generator 100 is worn on the forearm and the hand of the radiographer, the control mode related to controlling the X-ray source is set by using the user controller. Then, the switch controller 130 is gripped to operate X-ray generator 100 by the hand on which the X-ray generator 100 is worn. Then, a cone of the cone 110 of the X-ray generator 100 is arranged on a radiographing part of the target, and X-ray radiographing is performed by pressing the switch controller 130.

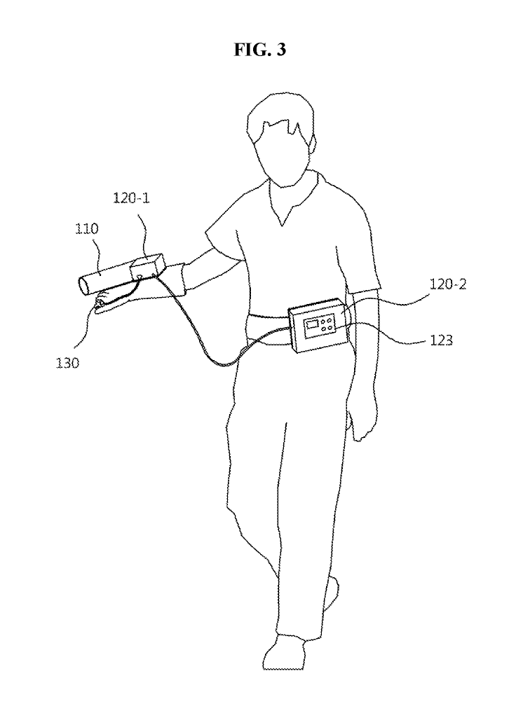

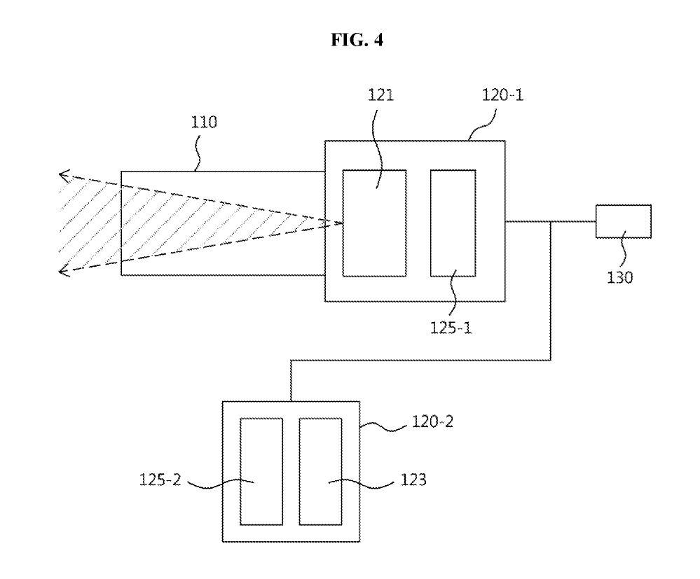

According to a wearable X-ray radiographing apparatus according to a second embodiment of the present invention, as schematically shown in FIGS. 3 and 4, an irradiator 120-1 and a controller 120-2 of an X-ray generator are formed to be physically separated from each other. In the second embodiment, the wearable unit includes a first wearable unit on which the irradiator 120-1 is mounted and which is worn on a first body part of the radiographer, and a second wearable unit on which the controller 120-2 is mounted and which is worn on a second body part of the radiographer. In addition, the switch controller 130 may be formed to be separated from the irradiator 120-1 and the controller 120-2, and may be connected to the irradiator 120-1 and the controller 120-2 in a wired or wireless manner.

In detail, the irradiator 120-1 may include the X-ray source 121 and the cone 110, and a second voltage booster 125-1. The controller 120-2 may include a battery and a first voltage booster 125-2, and the user controller 123. The irradiator 120-1 and the controller 120-2 that are separately formed as described above are connected to each other in a wired or wireless manner. Although it is not particularly limited, it is preferable to wirily connect the irradiator 120-1 and the controller 120-2 using a cable considering possibility of interference between wireless signals. A detailed configuration and operational principles thereof of the X-ray generator of the second embodiment is the same as those of the first embodiment except for the configuration of the irradiator 120-1 and the controller 120-2 that are separately formed, thus detailed descriptions thereof will be omitted.

In the X-ray radiographing apparatus of the second embodiment of the present invention, the irradiator 120-1 of the X-ray generator may be worn on the first body part of the radiographer, for example, on the hand and arm of the radiographer. The controller 120-2 may be worn on the second body part of the radiographer, for example, on an upper body such as waist, back, shoulder, flank, etc., or on a leg. In addition, the switch controller 130 is gripped by the radiographer's hand and is operated by being pressed by the radiographer. The wearable unit includes the first wearable unit to hold the irradiator 120-1 on the first body part, and the second wearable unit to hold the controller 120-2 on the second body part. Each of the wearable units may be configured with a holder for holding and a body that is worn on the body of the radiographer as the first embodiment. A detailed configuration of the wearable unit will be omitted since the configuration described in the first embodiment may be applied as it is. However, the second wearable unit may be a form such as band or suspenders, waistband, etc. that is capable of being worn on an upper body such as waist, back, shoulder, etc, or on a leg.

A usage method for the X-ray radiographing apparatus according to the second embodiment will be described.

First, the first wearable unit on which the irradiator 120-1 is mounted is worn by the radiographer on anywhere from his/her forearm(lower part of the arm) to his/her hand, and the second wearable unit on which the controller 120-2 is mounted is worn on the radiographer's waist. Herein, when the first wearable unit is configured with a gauntlet, the first wearable unit is worn by the radiographer inserting his/her hand into the first wearable unit and by pulling up the first wearable unit to his/her forearm just like wearing a glove. By wearing the first wearable unit as described above, the cone 110 of the irradiator 120-1 is placed on the hand of the radiographer.

Then, a control mode related to controlling the X-ray source 121 is set by using the user controller 123 of the controller 120-2.

The cone 110 of the X-ray radiographing apparatus is arranged on a radiographing part of the target, the radiographer grips the switch controller 130 with whichever hand the irradiator 120-1 is mounted, and X-ray radiographing is performed by pressing the switch controller 130.

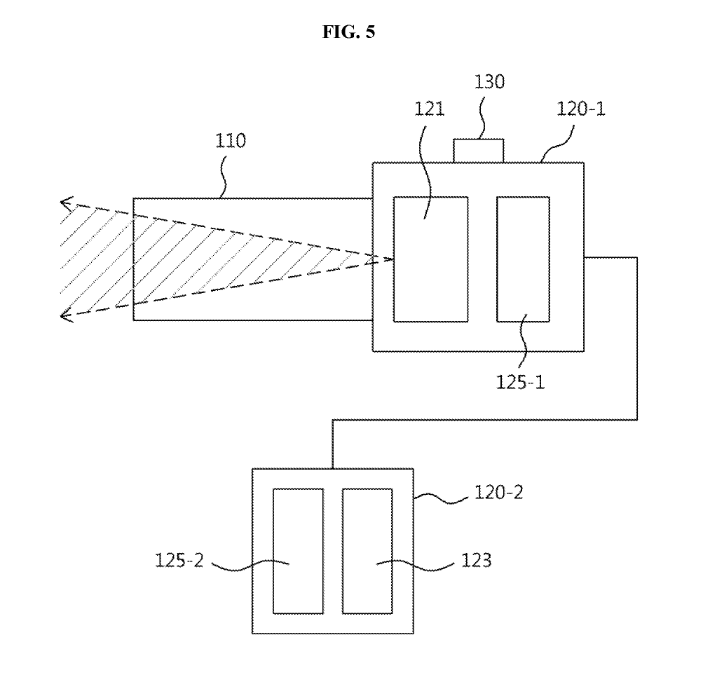

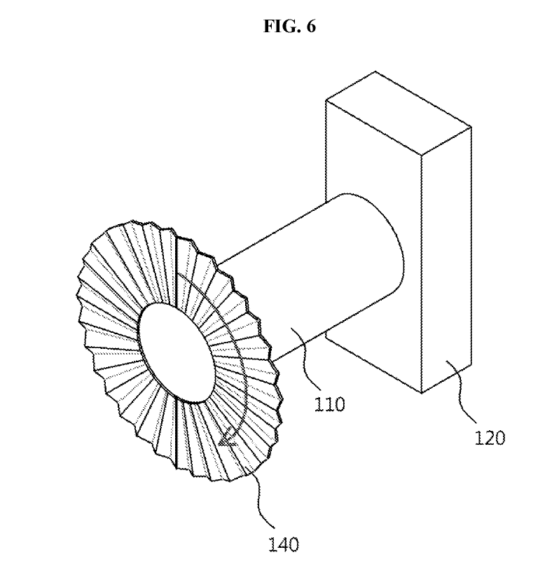

According to a wearable X-ray radiographing apparatus according to a third embodiment of the present invention, as schematically shown in FIG. 5, the X-ray generator is the same as that of the second embodiment in that the irradiator 120-1 and the controller 120-2 are formed to be physically separated from each other, but is different in that the switch controller 130 operating the X-ray generator is mounted on an external surface of a housing of the irradiator 120-1 to be integrated with the irradiator 120-1. In addition, the wearable unit is configured to be worn only on the body of the radiographer by providing the wearable unit only to the controller 120-2.

In other words, in the X-ray radiographing apparatus according to the third embodiment, the controller 120-2 is worn on the body of the radiographer, for example, on the waist, or on a hand and arm, the irradiator 120-1 is gripped by the radiographer's hand, and the X-ray generator is operated by pressing the switch controller 130 that is integrated with the irradiator 120-1. A detailed configuration part and operational principles thereof of other configurations of the X-ray generator and the wearable unit that are not described will be omitted since they are the same as those that are described in the first and second embodiments.

A usage method for the X-ray radiographing apparatus according to the third embodiment will be described.

First, the wearable unit on which the controller 120-2 is mounted is worn by the radiographer on his/her arm or a waist.

Then, a control mode related to controlling the X-ray source 121 is set by using the user controller 123 of the controller 120-2.

The irradiator 120-1 of the X-ray radiographing apparatus is gripped by the radiographer's hand, a cone of the cone 110 is ranged on a radiographing part of the target, and X-ray radiographing is performed by pressing the switch controller 130 that is integrated with the irradiator 120-1.

Meanwhile, the X-ray radiographing apparatus according to the first to the third embodiments may further include a radiation shielding membrane around an end part of the cone 110 that is heading for an X-ray irradiation direction. By installing the shielding membrane, it is possible to prevent the radiographer from being exposed to X-rays irradiated from the X-ray generator.

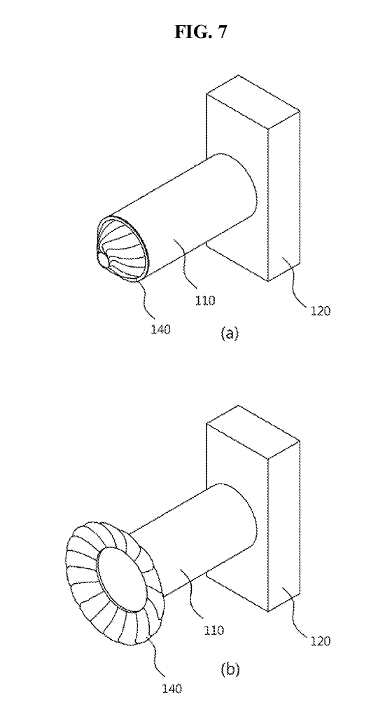

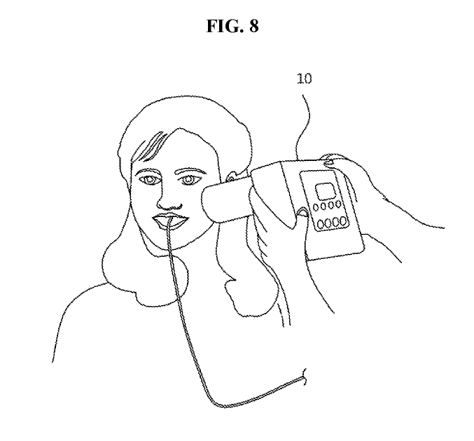

FIGS. 6 and 7 show examples of embodiments of the shielding membrane. The shielding membrane 140 shown in FIG. 6 exemplary shows a rotary open/close type shielding membrane. In addition, the shielding membrane 140 shown in FIG. 7 exemplary shows a folding type shielding membrane. FIGS. 6(a) and 6(b) respectively show a folded status and an unfolded status of the shielding membrane 140. Various methods commonly used in the related art may be applied to coupling structures for mounting the rotary open/close type shielding membrane and the folding type shielding membrane that are shown in the above on around the end part of cone 110.

In the present invention, a radiograph preparing path may be reduced and a damage caused by dropping the apparatus may be prevented by enabling the X-ray radiographing apparatus to be mounted on the body of a radiographer. In addition, since the apparatus is mounted on the body of the radiographer, stability of a radiographer's posture may be ensured even though the radiographer manipulates the apparatus by only one hand, and unexpected emergency situations may be immediately responded to.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A wearable X-ray radiographing apparatus, the apparatus comprising:
an X-ray generator including an X-ray source, a power supplier supplying driving power to the X-ray source, and a cone directing X-rays emitted from the X-ray source;
a switch controller controlled by a radiographer to operate the X-ray generator; and
a first wearable unit on which the switch controller is mounted and which is worn on a first body part of the radiographer,
wherein the X-ray generator further includes a user controller to control the X-ray generator.

2. The apparatus of claim 1, wherein the switch controller is physically separated from the X-ray generator and connected thereto in a wired or wireless manner.

3. The apparatus of claim 1, further comprising:
a holder holding the X-ray generator; and
a body configured with a shielding material to be worn on the body of the radiographer.

4. The apparatus of claim 1, a second wearable unit on which the X-ray generator is mounted and which is worn on a second body part of the radiographer.

5. The apparatus of claim 1, the first wearable unit includes a band to hold the switch controller on the first body part of the radiographer.

6. The apparatus of claim 4, wherein the second wearable unit is configured to be adapted to a hand of the radiographer.

7. The apparatus of claim 1, further comprising: a shielding membrane provided around the cone.

8. (canceled)

9. The apparatus of claim 1, wherein the switch controller further includes a battery and a first voltage booster.

10. The apparatus of claim 1, wherein the X-ray generator further includes a second voltage booster.
